(19) United States
(12) Patent Application Publication
(10) Pub. No.: US 2005/0069145 A1
(43) Pub. Date: Mar. 31, 2005

(54) Hearing protection

(76) Inventor: Ingvar Sjoqvist, Huddinge (SE)

Correspondence Address: HARNESS, DICKEY & PIERCE, P.L.C., P.O. Box 8910, Reston, VA, 20195, US

(21) Appl. No.: 10/797,598

(22) Filed: Mar. 11, 2004

(30) Foreign Application Priority Data
Mar. 14, 2003 (SE) 0300710-1

(51) Int. Cl. A61F 11/06; H04R 25/00

(52) U.S. Cl. 381/72; 381/371

(57) ABSTRACT

The present invention relates to an arrangement for preventing or at least reducing high acoustic pressure levels reaching the tympanic membrane of an ear. In particular, the present invention relates to an arrangement for preventing or at least reducing high acoustic pressure levels reaching the tympanic membrane of an ear by means of placing a protector in an external auditory canal of said ear. The ear protector comprises members for easily engaging or disengaging the ear protector.

### TECHNICAL FIELD

The present invention relates to an arrangement for preventing or at least reducing high acoustic pressure levels reaching the tympanic membrane of an ear. In particular, the present invention relates to an arrangement for preventing or at least reducing high acoustic pressure levels reaching the tympanic membrane of an, ear by means of placing a protector in an external auditory canal of said ear.

### BACKGROUND OF THE INVENTION

In today's environment, human beings and animals live under a constant bombardment of noise. The noise levels, or the acoustic pressure measured in decibels, are often extremely high, so high in fact that they can damage hearing.

Impaired hearing can be caused by sudden loud noises, or from having been exposed to high levels of noise for a relatively long time. Noise with an acoustic pressure of over 80-85 dB is usually considered to cause impaired hearing if the ear is exposed to these levels for quite some time.

To avoid damage to hearing in environments where there are high noise levels, a large number of different ear protectors have been developed. In principle, there are two different types of ear protectors, the first type surrounding the whole of the pinna, and the second type being inserted into the external auditory canal and closing it.

The second type often requires shaping it with the fingers before inserting it into the ear. This means that it is necessary to use both hands to apply the ear protector and also that dirt and bacteria on the hands and fingers risk getting into the ear. For workers who use relatively thick protective gloves, it is difficult to apply the protector without taking off said gloves.

Others require screwing the protector in, or gripping the pinna in order to more easily insert the protector.

There is therefore a need for an ear protector which is easy to apply in the external auditory canal of an ear and which provides good protection against high levels of noise.

For ear protectors of the first type, total enclosure of the pinna may feel uncomfortable and awkward. A tight arrangement like this can be uncomfortable especially in warm weather.

### DISCLOSURE OF THE INVENTION

The main object of the present invention is therefore to make available an ear protector which prevents or at least reduces the abovementioned problems.

A particular object of the present invention is to make available a method and an apparatus which is comfortable, gives good protection against high levels of noise and is easy to control in the sense of whether protection is required or not at a given time.

The abovementioned objects are achieved by a method and equipment in accordance with the attached independent claims.

Advantageous alternatives and embodiments of the present invention are afforded by means of methods and equipment according to the atached dependent claims.

An advantage of the present invention is that it is possible to remove the ear protector by a simple manoeuvre and then reapply the protector by a simple manoeuvre.

Further features and advantages of the invention will become clear from the following detailed description of embodiments according to the invention.

### BRIEF DESCRIPTION OF THE DRAWINGS

The present invention will be better understood from the following detailed description of preferred embodiments according to the present invention and from attached FIGS. 1 to 3, which are intended only to illustrate the preferred embodiments and not intended to be restrictive for the present invention.

FIG. 1 shows a preferred embodiment of the invention in a perspective view.

FIG. 2 shows the arrangement from FIG. 1 in a side view.

FIG. 3 shows a detail of the arrangement from FIG. 1.

### PREFERRED EMBODIMENTS

The description below details specific techniques and applications in order to provide a clear understanding of the present invention. However, it will be obvious to the person skilled in the art that the present invention can be implemented in other embodiments which differ from what has been indicated in detail herein. Detailed descriptions of well known methods and equipment have been omitted so as not to fill the description of the present invention with unnecessary details.

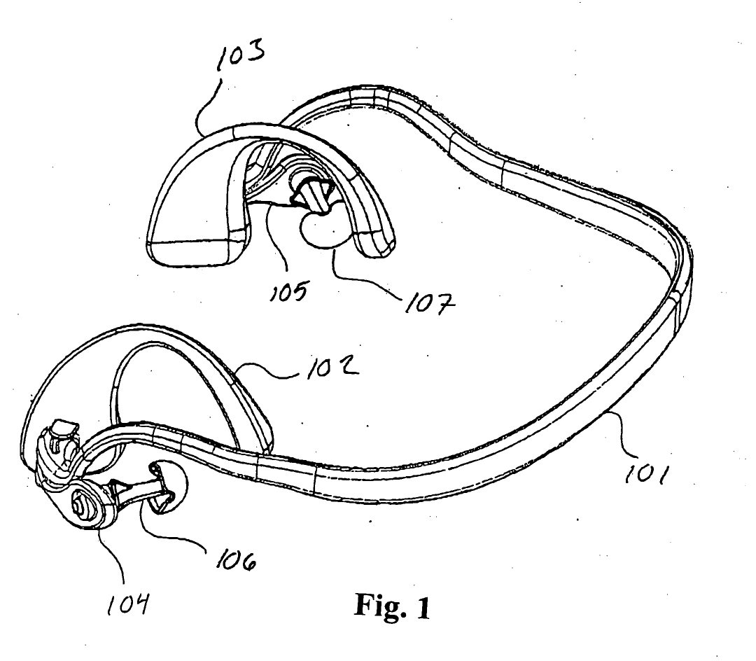

FIG. 1 shows a perspective view of a preferred embodiment according to the invention. The arrangement consists of a first part 101, which is a bow intended to fit round the head of a person wearing the ear protector, and a second part 102 and a third part 103. The second and third parts 102 and 103 are intended to bear against the skull around and behind an ear. The second and third parts 102 and 103 secure the ear protector in the right position on the skull.

In an alternative embodiment, the first part 101 is pretensioned by a spring and the second and third parts consist of plates which, with the aid of the spring pretensioning, press on opposite sides of the head in order in this way to secure the ear protector.

A fourth part 104 and a fifth part 105 are arranged pivotably on the ear protector and are shown in FIG. 1 in a first, outward position. When a person is wearing the ear protector and the fourth and fifth parts are in the first position, the ear protector does not suppress noise, and, for example, the person is therefore able to talk with someone.

The fourth and fifth parts comprise ear plugs 106 and 107 which are exchangeable and which, when the fourth and/or fifth part assume(s) a second position, fit into the auditory canal of a respective ear. The ear plugs thus suppress noise and the ear protector protects the wearer from high acoustic pressures. The ear plugs can have a number of different designs.

The fourth and fifth parts function independently of one another. Thus, a wearer can choose to protect just one ear from loud noise, for example if noises are coming from a specific direction.

With the design according to the invention, it is possible for the wearer, without taking the actual ear protector off, to cut out the noise suppression by moving the fourth and/or fifth parts to the first position. In this way the user can, for example, communicate with co-workers.

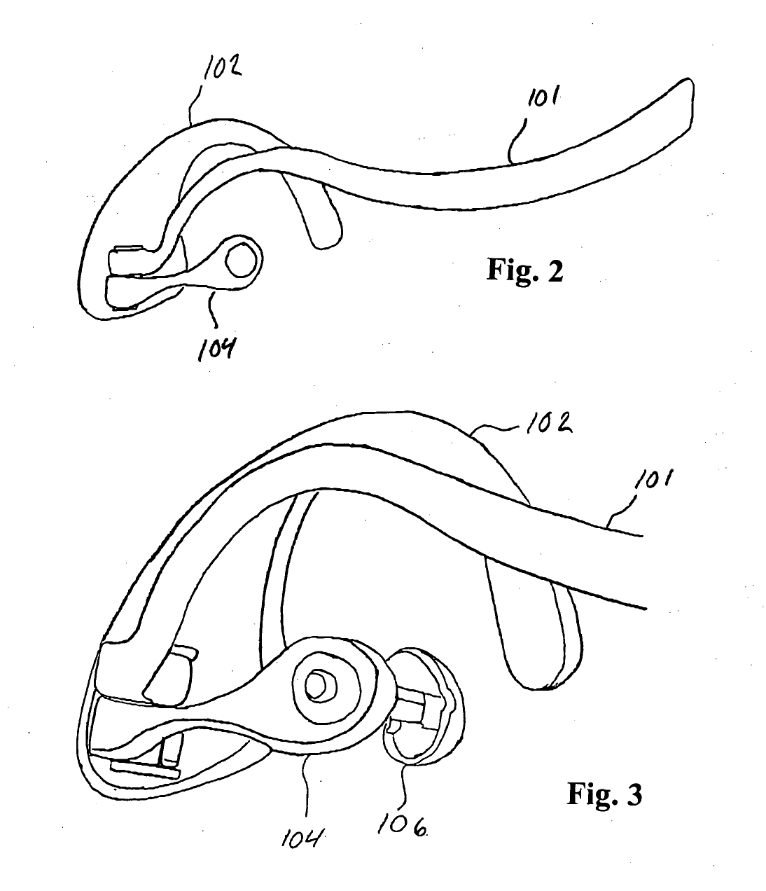

FIG. 2 shows the arrangement from FIG. 1 in a side view. The same details are indicated by the same reference numbers. FIG. 3 shows an enlarged detail of part of the ear protector from FIG. 1 in a perspective view when the fourth part 104 is situated in the second, inward position.

The fourth and fifth parts can be pretensioned by a spring so that the ear plug is pressed in towards the auditory canal when the fourth and fifth parts are situated in the second, inward position.

According to a further embodiment of the invention, the fourth and fifth parts are pretensioned by a spring so that a slight pressure on said fourth or fifth part, or on an arrangement which triggers the spring, releases the fourth and fifth parts so that these assume the first, outward position.

It is obvious that the present invention can be varied in many different ways. Such variations are not to be regarded as a departure from the scope of the present invention. All such modifications which are obvious to the skilled person are intended to be included in the scope of the attached patent claims.

1. Ear protector arranged to protect a first ear and a second ear from loud noise, comprising a bow which has a first part connecting a second part and a third part,
said second part comprising at least a first contact point to be applied in proximity to said first ear,
said third part comprising at least a first contact point to be applied in proximity to said second ear, characterized in that
the ear protector comprises a fourth part arranged rotatably on said second part and comprising a first ear plug which fits into said first ear to suppress noise,
the ear protector comprises a fifth part arranged rotatably on said third part and comprising a second ear plug which fits into said second ear to suppress noise,
said fourth part is designed to be able to assume a first position in which said ear plug is in a position outside said first ear, and a second position in which said ear plug is in a position which fits in said ear in order thereby to suppress noise,
said fifth part is designed to be able to assume a first position in which said ear plug is in a position outside said auditory canal, and a second position in which said ear plug is in a position in said auditory canal in order thereby to suppress noise.

2. Ear protector according to claim 1, in which
said fourth and fifth parts fit into a respective auditory canal of the respective said ear.

3. Ear protector according to claim 1, in which
said second and third parts are arranged to pass round the pinna of an ear and in this way hold said ear protector securely in a position such that said fourth and fifth parts, in the respective second position, fit said ear plugs into said auditory canals.

4. Ear protector according to claim 1, in which
said first part is pretensioned by a spring so that said second and third parts press towards a position in proximity to said ears in order to hold said ear protector securely in a position such that said fourth and fifth parts, in the respective second position, fit said ear plugs into said auditory canals.

5. Ear protector according to claim 1, in which
said fourth and fifth parts are pretensioned by a spring so that, in said second position, they exert a pressure inwards towards said auditory canal.

6. Ear protector according to claim 1, in which
said fourth and fifth parts are pretensioned by a spring in said second position so as to assume said first position under a slight pressure.

7. Ear protector according to claim 1, in which
said ear protector comprises a radio, mobile telephone, pager or other electronic equipment and devices for conveying sound or a signal in an audible manner through said ear plugs.

8. Ear protector according to claim 1, in which
said ear plugs can be exchanged from said fourth and fifth parts.
